(19) United States
(12) Patent Application Publication
(10) Pub. No.: US 2026/0216004 A1
(43) Pub. Date: Jul. 30, 2026

(54) SMART PACK WITH OLEDS

(71) Applicant: JANSSEN PHARMACEUTICA NV, BEERSE (BE)

(72) Inventors: JASON TANSKY, COLLEGEVILLE, PA (US); MARISA EHINGER, LAMBERTVILLE, NJ (US)

(21) Appl. No.: 19/152,139

(22) PCT Filed: Feb. 9, 2024
(86) PCT No.: PCT/IB2024/051204

§ 371 Date: Jul. 30, 2025

Related U.S. Application Data
(60) Provisional application No. 63/484,480, filed on Feb. 10, 2023.

(51) Int. Cl. A61J 7/04; A61J 1/03; G16H 20/13; H10K 59/10

(52) U.S. Cl. CPC A61J 7/0436; A61J 1/035; G16H 20/13; H10K 59/10; A61J 2205/60

(57) ABSTRACT

Provided herein is a reusable smart pill dispenser for assisting a user to take medication stored in a disposable blister package. The pill dispenser can comprise a plurality of apertures that align with each dose of a disposable blister package of medication, a plurality of illuminating regions associated with the plurality of apertures of the pill dispenser, and a controller configured to control the illuminating regions and a sensor to read an identifier on the disposable blister package. The controller can be configured to control at least one of the plurality of illuminating regions associated with at least one of the plurality of apertures based on the identifier. Other features and aspects, as well as a method for assisting a user to take medication stored in a disposable blister package using a reusable pill dispenser, are described.

### FIELD

The present invention relates to a reusable pill dispenser, and more specifically to a reusable smart pill dispenser that provides patient alerts for administering medication stored in a disposable blister package.

### BACKGROUND

A medication prescription for a patient often includes a dosage regimen for the patient that dictates the dosage required, the timing and frequency the dosage should be ingested, and instructions for use, such as to ingest the dosage with food or avoid certain activities after ingestion. When a patient fails to adhere to their prescription (often called medication non-adherence), a variety of issues can arise. For example, if a patient misses a single dose, their body will no longer have an effective amount of the medication, which can cause the patient to become sicker or prolong the term of their illness. Missing a dose or multiple dosages of a given medication can also cause the patient's body to develop a resistance to the medication, or trigger undesirable side effects such as withdrawal symptoms. In the context of clinical research, medication non-adherence can lead to underestimations of medication efficacy, study variance, lower study power, and safety concerns.

Failure to adhere to a prescription can be caused by a variety of reasons. For instance, a patient may simply forget to take a dose or may not understand the directions of their prescription. This problem can be exacerbated for patients with dementia and other memory related disorders, who may further forget that they already took a dose and take another dose too early. Moreover, certain prescriptions and/or clinical trials may require a patient to take increasing or decreasing doses of the medication over time (e.g., ascending or descending dosage regimens, also called uptitration and downtitration), such as one dose on the first day, three doses on the second day and so on, or a gradual increase or decrease in a dose, which may confuse the patient and lead him or her to take too few or too many doses and/or medication.

One method drug distributors rely on to encourage patients to take the proper dosage according to their prescription is to include printed instructions on the medication package itself that provide the prescription information. One type of medication package relied on is a large cardboard shell that surrounds a blister package with doses of the medication. Including prescription information printed on medical packaging presents a number of issues. For example, the drug distributor of an ascending or descending dosage regimen must carry multiple stocks of dosage packs with instructions for each stage of the dosage regimen, and patients must utilize the appropriate dosage pack based on the stage of the dosage regimen they are in. In addition to requiring the drug distributor to store a larger stock of dosage packs, this also introduces the risk that a dosage pack with the wrong instructions may be distributed too early or too late for a given patient with respect to the stage of the dosage regimen they are in and/or that the patient may use the incorrect dosage pack at a given time. Further, changing dosage packs can lead to unused drug product that must be destroyed, such as when the patient is progressing to a new stage of a trial and must follow the instructions of a second medication package.

A number of products are also available to patients to encourage patients to take their medications as prescribed. For example, a variety of pill containers are available that indicate to a patient the proper dosage of a given medication for a set period, with receptacles for each day of the period to receive the medication the patient should consume. However, these pill containers require the patient manually add their medication to each receptacle, and then remember to take the medication at the right time. Timer caps for pill bottles are also available, which measure the time elapsed since the patient last opened the pill bottle and may provide an alarm that automatically indicates when the patient should take the next dose based on the time elapsed. Timer caps and other similar products, however, are suited only to providing indications based on timing, and fail to provide indications to patients regarding the proper dosage the patient should take.

### SUMMARY

Provided herein is a reusable smart pill dispenser that provides guidance to a patient in the form of alerts to facilitate patient adherence to their dosage regimen. The pill dispenser includes a plurality of apertures that align with each dose of a disposable blister package of medication. The pill dispenser includes a plurality of illuminating regions associated with the plurality of apertures of the pill dispenser. The pill dispenser includes a controller configured to control the illuminating regions and a sensor to read an identifier on the disposable blister package. The identifier can contain information the controller uses to determine how many doses of medication the patient should take as well as the frequency and timing of those doses. After taking all the medication in a given disposable blister package, the patient can dispose of that blister package and insert a new blister package into the reusable pill dispenser, which then reads the identifier of the new blister package and can control the illuminating regions according to the instructions of the identifier of the new blister package. Accordingly, a pill dispenser according to one or more of the embodiments disclosed herein can eliminate the need to provide detailed prescription information on a medication package containing a blister package, which, among other benefits, can reduce material costs and labor costs associated with packaging blister packages into medication packages (such as cardboard shells).

Upon reading an identifier of a given blister package, the controller can control the illuminating region associated with the aperture of the reusable pill dispenser that contains a dose that is due, thereby providing a visual cue to the patient to take that specific dose of medication. The controller can determine not only when a given dose is due, but also how many doses are due at a given time, thereby providing guidance to the patient regarding when to take a dose and which doses to take at that time.

In one or more examples, the pill dispenser can include a user compliance mechanism for accepting an indication that the patient took a dose of the blister package. The pill dispenser can also be communicatively connected to an external communication device, with the pill dispenser configured to both communicate information about the patient's indication that he or she took a dose as directed or the patient's failure to do so, as well as receive information related to updating the device, updating the patient's information, updating the dosage regimen, etc.

The pill dispenser can include a communication chip such that the reusable pill dispenser is communicatively connected to an external device. Using the communication chip, the reusable pill dispenser can transmit messages to and receive messages from the external device. The controller of the reusable pill dispenser can control the illuminating regions of the reusable pill dispenser based on messages received from the external device. For example, the reusable pill dispenser can receive information related to the patient's dosage regimen, determine an updated dosage regimen, and then control illuminating regions of the reusable pill dispenser based on the updated dosage regimen. The communication chip can also relay adherence information regarding which doses the patient acknowledged via the user compliance mechanism, as well as which doses the patient failed to acknowledge via the user compliance mechanism. Thus, a pill dispenser according to one or more of the embodiments disclosed herein can direct the patient to adhere to an updated dosage regimen without requiring the user begin a new blister package, thereby avoiding the destruction of unused drug product.

Accordingly, a reusable pill dispenser according to one or more of the embodiments disclosed herein can assist patients with adhering to their prescription for medication by providing alerts to indicate when a dose is due, and indicating what dose is due at that time without requiring the patient to determine when a dose is due or what dose is due, rely solely on printed instructions, or manually add medication to receptacles of the pill dispenser. Moreover, a pill dispenser according to one or more of the embodiments disclosed herein can remove the necessity for drug manufacturers to store a large stock of dosage packs with different printed instructions on each and reduce the risk that a dosage pack with the wrong printed instructions will be delivered to a patient. A reusable pill dispenser according to one or more of the embodiments disclosed herein can enable medication administrators to update the patient's dosage by communication dosage updates to the reusable pill dispenser and track the patient's medication adherence based on messages generated by the reusable pill dispenser.

In one or more examples, a reusable pill dispenser for assisting a user to take medication stored in a disposable blister package can comprise: a plurality of apertures, wherein each of the plurality of apertures is arranged to align with a dose of the disposable blister package, a plurality of illuminating regions, wherein at least one of the plurality of illuminating regions is associated with at least one of the plurality of apertures, a sensor to read an identifier on the disposable blister package, and a controller configured to control the at least one of the plurality of illuminating regions that is associated with the at least one of the plurality of apertures based on the identifier.

The controller can be configured to determine a dosage regimen based on the identifier. The controller may be configured to control the at least one of the plurality of illuminating regions that is associated with the at least one of the plurality of apertures based on the determined dosage regimen. The dosage regimen can be based in part on a dosage database corresponding to the identifier.

The reusable pill dispenser can comprise a user compliance mechanism for accepting an indication that the user has taken at least one dose of the disposable blister package. The controller can be configured to control the at least one of the plurality of illuminating regions that is associated with the at least one of the plurality of apertures based on the indication. Controlling the at least one of the plurality of illuminating regions that is associated with the at least one of the plurality of apertures may comprise: controlling the at least one of the illuminating regions that is associated with the at least one of the plurality of apertures in a first mode, and if a predetermined period passes without receiving the indication, controlling the at least one of the illuminating regions that is associated with the at least one of the plurality of apertures in a second mode. The indication may specify which doses of the disposable blister package the user has taken and the controller can be configured to trigger an alarm if the at least one dose of the disposable blister package the user has taken is contrary to a dosage regimen for the user. The user compliance mechanism can be one of a push button, an electronic trigger, and a switch.

The reusable pill dispenser can comprise a communication device to communicate with an external device. The communication device can be operative to receive update information from the external device related to one or more of a device update, patient information update, and a dosage regimen update. The controller may be configured to determine an updated dosage regimen based at least in part on the update information. The communication device can be configured for transmitting and receiving messages over one or more cellular networks. The communication device may be configured for transmitting and receiving messages using short message service (SMS). The communication device may be configured for transmitting and receiving messages over a Wi-Fi network.

The identifier of reusable pill dispenser can comprise one or more of a serial number for the disposable blister package, a name of the medication stored in the disposable blister package, patient information corresponding to the user, an expiration of the medication stored in the disposable blister package, and a prescription for the medication stored in the disposable blister package. Controlling the at least one of the plurality of illuminating regions that is associated with the at least one of the plurality of apertures can comprise illuminating the at least one illuminating region when a dose of the disposable blister package is due to be taken. The sensor can be a radio frequency identification (RFID) reader. The plurality of illuminating regions can comprise one or more light emitting diodes (LEDs). The reusable pill dispenser may comprise a screen that comprises the plurality of apertures and the plurality of illuminating regions, wherein each of the plurality of illuminating regions comprises one or more LEDs integrated into the screen that are positioned adjacent to a respective one of the plurality of apertures. The reusable pill dispenser may comprise a display for communicating information to the user. The display can be an E-ink display.

The reusable pill dispenser may comprise a first portion and a second portion connected by a hinge, wherein the first portion and the second portion contact one another in a closed position. The reusable pill dispenser can comprise a latch to secure the reusable pill dispenser in the closed position. The reusable pill dispenser may comprise a plurality of spring clips to retain the disposable blister package inside the reusable pill dispenser.

A method for assisting a user to take medication stored in a disposable blister package using a reusable pill dispenser can comprise: reading an identifier on the disposable blister package via a sensor of the reusable pill dispenser, the reusable pill dispenser comprising a plurality of apertures, wherein each of the plurality of apertures is arranged to align with a dose of the disposable blister package, and controlling at least one illuminating region of a plurality of illuminating regions of the reusable pill dispenser based on the identifier, wherein at least one illuminating region of the plurality of illuminating regions is associated with at least one of a plurality of apertures of the reusable pill dispenser.

The method may comprise determining a dosage regimen based on the identifier. The method may comprise controlling the at least one illuminating region of the plurality of illuminating regions of the reusable pill dispenser based on the determined dosage regimen.

Determining the dosage regimen can be based in part on a dosage database corresponding to the identifier.

The method may comprise receiving an indication from a compliance mechanism of the reusable pill dispenser. The method may comprise controlling at least one of the plurality of illuminating regions of the reusable pill dispenser based on the received indication. The method may comprise controlling the at least one of the illuminating regions in a first mode, and if a predetermined period passes without receiving the indication, controlling the at least one illuminating region of a plurality of illuminating regions of the reusable pill dispenser in a second mode. The indication may specify which doses of the disposable blister package the user has taken and the method can include triggering an alarm if the at least one dose of the disposable blister package the user has taken is contrary to a dosage regimen for the user. The compliance mechanism can be one of a push button, an electronic trigger, and a switch.

The method can comprise receiving update information via a communication device of the reusable pill dispenser. The update information may be related to one or more of a device update, patient information update, and a dosage regimen update. The method can comprise determining an updated dosage regimen based at least in part on the received update information. The update information can be received via one or more messages received over one or more cellular networks. The update information may be received via one or more messages using short messaging service (SMS). The update information may be received via one or more messages received over a Wi-Fi network.

The identifier can be one or more of a serial number for the disposable blister package, a name of the medication stored in the disposable blister package, patient information corresponding to the user, an expiration of the medication stored in the disposable blister package, and a prescription for the medication stored in the disposable blister package. Controlling the at least one illuminating region of a plurality of illuminating regions of the reusable pill dispenser may comprise illuminating the at least one illuminating region when a dose of the disposable blister package is due to be taken. The sensor can be a radio frequency identification (RFID) reader. The plurality of illuminating regions can comprise one or more light emitting diodes (LEDs). The reusable pill dispenser may comprise a screen that comprises the plurality of apertures and the plurality of illuminating regions, wherein each of the plurality of illuminating regions comprises one or more LEDs integrated into the screen that are positioned adjacent to a respective one of the plurality of apertures. The method can comprise displaying information via a display of the reusable pill dispenser. The display may be an E-ink display.

The reusable pill dispenser can comprise a first portion and a second portion connected by a hinge, wherein the first portion and the second portion contact one another in a closed position. The reusable pill dispenser may comprise a latch to secure the reusable pill dispenser in the closed position. The reusable pill dispenser can comprise a plurality of spring clips to retain the disposable blister package inside the reusable pill dispenser.

Additional advantages will be readily apparent to those skilled in the art from the following detailed description. The aspects and descriptions herein are to be regarded as illustrative in nature and not restrictive. It will be appreciated that any of the variations, aspects, features, and options described in view of the systems apply equally to the methods and vice versa. It will also be clear that any one or more of the above variations, aspects, features, and options can be combined.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

The invention will now be described, by way of example only, with reference to the accompanying drawings, in which:

FIG. 1 shows a block diagram of an exemplary smart pack system, according to one or more examples of the disclosure;

FIG. 2 shows an exemplary reusable pill dispenser, according to one or more examples of the disclosure;

FIG. 3 shows an exemplary disposable blister package, according to one or more examples of the disclosure;

FIG. 4 shows an exemplary method for administering medication via a reusable pill dispenser based on an identifier of a disposable blister package, according to one or more examples of the disclosure;

FIG. 5 shows an exemplary method for administering medication via a reusable pill dispenser based on a received indication, according to one or more examples of the disclosure;

FIG. 6 shows an exemplary method for administering medication via a reusable pill dispenser based on received update information, according to one or more examples of the disclosure;

FIG. 7A shows an exploded view of an exemplary reusable pill dispenser, according to one or more examples of the disclosure;

FIG. 7B shows a perspective view of an assembled reusable pill dispenser, according to one or more examples of the disclosure;

FIG. 8 shows a cutaway internal view of the exemplary reusable pill dispenser of FIG. 7A, according to one or more examples of the disclosure.

FIG. 9A shows a first side view of an exemplary reusable pill dispenser, according to one or more examples of the disclosure;

FIG. 9B shows an opposing side view of the exemplary reusable pill dispenser of FIG. 9A, according to one or more examples of the disclosure;

FIG. 9C illustrates a perspective view of the reusable pill dispenser of FIG. 9A, according to one or more examples of the disclosure; and

FIG. 10 shows an exemplary computing device, according to one or more examples of the present disclosure.

### DETAILED DESCRIPTION

In the following description of the various examples, reference is made to the accompanying drawings, in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art and the generic principles herein may be applied to other examples. Thus, the present invention is not intended to be limited to the examples shown but is to be accorded the widest scope consistent with the principles and features described herein.

Described herein is a reusable pill dispenser that provides patient alerts to assist a patient to take medication stored in a disposable blister package. The reusable pill dispenser provides guidance to the patient in the form of alerts, such as visual cues, auditory cues, tactile cues, etc., to facilitate patient adherence to their dosage regimen for the medication stored in the disposable blister package. The reusable pill dispenser includes a plurality of apertures and a plurality of illuminating regions associated with the apertures. The apertures of the reusable pill dispenser are arranged to align with corresponding doses of the disposable blister package. The reusable pill dispenser includes a computer processing unit (CPU) (e.g., a controller) configured to control the illuminating regions of the reusable pill dispenser according to the patient's dosage regimen to assist the patient taking their medication.

The CPU of the reusable pill dispenser can include or be operatively connected to a sensor that reads an identifier of the disposable blister package, with the identifier providing information relating to the patient's dosage regimen. Upon reading an identifier of the disposable blister package, the CPU can be configured to determine the patient's dosage regimen.

The CPU of the reusable pill dispenser can include or be operatively connected to a communication chip that is in turn communicatively connected to an external device. Via the communication chip, the CPU can transmit messages to and/or receive messages from the external device. The messages can relate to update information that is relevant to the patient's dosage regimen, such as information indicating the patient's dosage regimen has changed, information that relevant patient information has changed which affects the patient's dosage regimen, etc.

The CPU of the reusable pill dispenser can control the illuminating regions of the reusable pill dispenser to provide alerts to the patient when a dose is due to be taken. The CPU can control the specific illuminating region(s) that correspond to the aperture or apertures that contain dose(s) the patient is due to take at each time. The CPU can control the illuminating regions of the reusable pill dispenser based on the identifier of the disposable blister package and/or information received from an external device.

The reusable pill dispenser can include a compliance mechanism for receiving an indication from the user. For instance, the compliance mechanism can receive an indication from the patient that they took a dose or dose(s) from the disposable blister package. The CPU of the reusable pill dispenser can be communicatively connected to the compliance mechanism such that the CPU receives the indication, and can be configured to control the illuminating regions of the reusable pill dispenser based on the indication. Upon receiving an indication, the CPU may control the illuminating regions of the reusable pill dispenser, such as by turning them off. If an indication is not received, the CPU may control the illuminating regions of the reusable pill dispenser to provide an alternative indication that a dose is due, such as by controlling the illuminating regions of the reusable pill dispenser to flash intermittently. If an indication is not received for a predetermined time, the CPU may control the illuminating regions to turn off. In one or more examples, the CPU may communicate information relating to the patients adherence to their dosage regimen (e.g., based on receiving an indication from the compliance mechanism or the absence of receiving an indication) to the external device.

As used herein, the singular forms “a,” “an,” and “the” used in the following description are intended to include the plural forms as well unless the context clearly indicates otherwise. It is to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

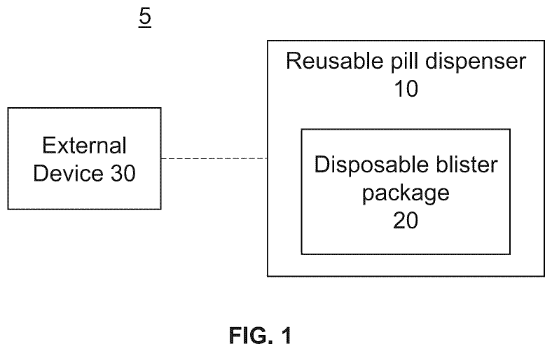

FIG. 1 shows a block diagram of an exemplary smart pack system 5, according to one or more examples of the disclosure. As shown, the smart pack system 5 includes a reusable pill dispenser 10, a disposable blister package 20, and an external device 30. In one or more examples, the smart pack system 5 can provide indications that assist a user (e.g., a patient or a health care provider (HCP)) to administer medication stored in the disposable blister package 20 according to a dosage regimen for that user.

The disposable blister package 20 can include a number of doses of medication stored in domes (also called “blisters”) of the disposable blister package 20. Each dome can be a dome-like structure that is sized to receive a dose of medication within the dome and secured there by a base of the disposable blister package 20 (such as a foil base). The reusable pill dispenser 10 can receive the disposable blister package 20 and provide indications to the user of the reusable pill dispenser 10 that assist the user with taking medication stored in the disposable blister package 20. For example, the reusable pill dispenser 10 can include illuminating regions and control the illuminating regions to provide visual cues to the user that indicate the user should take one or more doses of the disposable blister package 20. In one or more examples, the reusable pill dispenser 10 can provide other types of indications, such as auditory indications by emitting a sound from a speaker (e.g., a beeping noise, pre-recorded instructions, and/or an alarm), tactile indicators by vibrating the reusable pill dispenser 10, etc.

In one or more examples, the reusable pill dispenser 10 can be shaped like a foldable “wallet.” The reusable pill dispenser 10 can include a hinge on one side with latch mechanism(s) on the side of the wallet that opposes the hinge. The reusable pill dispenser 10 can store the disposable blister package 20 inside the wallet such that the doses of the disposable blister package 20 are securely stored in a convenient mobile container. The reusable pill dispenser 10 can be composed of a material that is capable of withstanding impact falls as well as mild corrosive substances, such as a polymeric material. The reusable pill dispenser 10 can be composed of a rigid material or a flexible material.

In one or more examples, the reusable pill dispenser 10 can be communicatively connected to the external device 30. For instance, the reusable pill dispenser 10 can be connected via a wireless communication method such as Bluetooth to the external device 30 and configured to both transmit and receive messages to and from the external device 30. The external device 30 can be a cloud-based device, a smart phone device of the user of the reusable pill dispenser 10, a smart phone device of another individual, a computing device of a prescribing physician or other medical administrator, etc. As will be described further below, in one or more examples, the external device 30 can communicate certain information to the patient and/or to the reusable pill dispenser 10 regarding the user's dosage regimen.

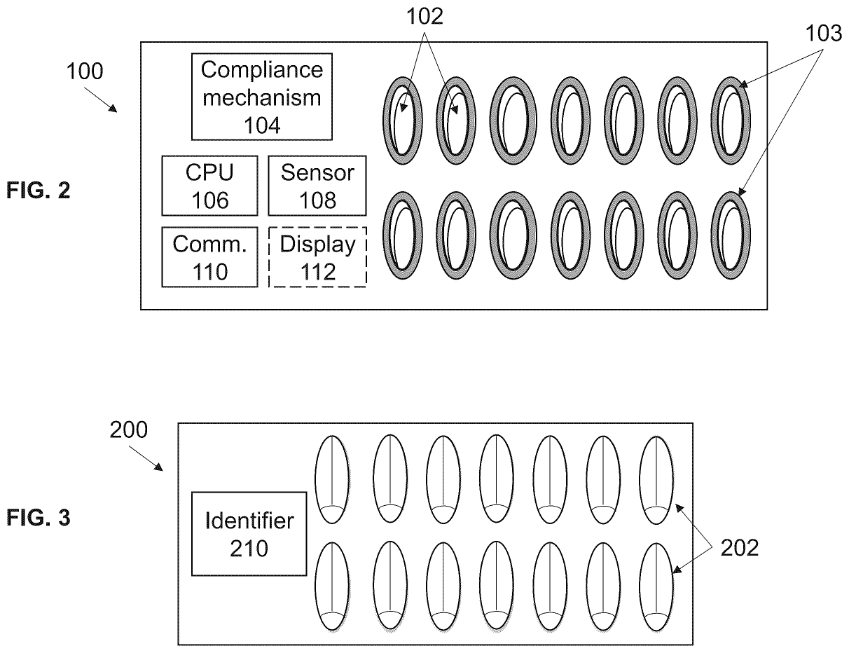

FIG. 2 shows an exemplary reusable pill dispenser 100, according to one or more examples of the disclosure. The reusable pill dispenser 100 can be used in the smart pack system described above. In one or more examples, the reusable pill dispenser 100 can receive a disposable blister package and provide indications (e.g., visual cues, auditory cues, etc.) that assist a user to take medication stored in the disposable package. The reusable pill dispenser 100 can include a number of apertures 102 surrounded by a number of illuminating regions 103, a compliance mechanism 104, a computer-processing unit (CPU) 106, a sensor 108, and a communication device 110.

The apertures 102 of the reusable pill dispenser 100 can be configured to receive doses of a disposable blister package, as will be discussed further below. The apertures 102 may extend through the reusable pill dispenser 100. Alternatively, the reusable pill dispenser 100 may include a top layer and a bottom layer, with the apertures extending only through the top layer. In such scenario, the reusable pill dispenser may include a space between the top layer and the bottom layer to receive a disposable blister package.

The illuminating regions 103 of the reusable pill dispenser 100 can be associated with the apertures 102 of the reusable pill dispenser 100. As shown in FIG. 2, each aperture 102 has a corresponding illuminating region 103 associated with the aperture 102. Optionally, the reusable pill dispenser 100 can be configured with fewer illuminating regions 103, such that not every aperture 102 has an associated illuminating region 103. The illuminating regions 103 of the reusable pill dispenser 100 can be, for example, light emitting diodes (LEDs) or organic light emitting diodes (OLEDs). The illuminating regions 103 can be configured to emit the same color of light or different colors of light.

The compliance mechanism 104 can accept an indication from a user. For instance, the compliance mechanism 104 can accept an indication from the user that the user has taken a dose of a disposable blister package inserted in the reusable pill dispenser 100. The user can activate the compliance mechanism 104 after taking a given dose of the disposable blister package, thereby providing an indication to the reusable pill dispenser 100 that the user ingested the dose. The compliance mechanism can, for example, be one of a push button, a switch, an electronic trigger, etc. In one or more examples, the compliance mechanism 104 can be incorporated into the structure of the reusable pill dispenser 100 without requiring the user to affirmatively provide the indication. For example, the compliance mechanism 104 can include an electrical trigger (e.g., a wire, electrical trace, or thin film circuit) that extends over each aperture 102 of the reusable pill dispenser 100 that automatically triggers an indication that is communicated to the CPU 106. For instance, where the compliance mechanism 104 includes a wire, the wire can break when the user removes a dose from a blister dome located within a aperture 102, and the breaking of the wire can trigger the indication. In one or more examples, the compliance mechanism 104 can include an electrical trigger that extends along the underside of the reusable pill dispenser 100 that operatively connects to the base of a disposable blister package (e.g., a foil backing of a disposable blister package) such that a dose being removed from the disposable blister package through the base of the disposable blister package actuates the electrical trigger.

In one or more examples, the compliance mechanism 104 may trigger an alarm if the patient fails to adhere to their dosage regimen properly (e.g., if the user has taken a dose that is contrary to their dosage regimen). For example, if the reusable pill dispenser 100 receives a disposable blister package that includes doses of medication that should be ingested in a specific sequence (e.g., an uptitration or downtitration regimen with multiple different pills having different dose amounts, or a regimen having distinct pills with different active ingredients), the CPU 106 may detect that the user has taken the wrong dose from the disposable blister package after receiving an indication from the compliance mechanism 104 that the user removed a dose from the reusable pill dispenser 100. The CPU 106 may detect that the user has taken too few or too many doses from the disposable blister package after receiving an indication from the compliance mechanism 104. Triggering an alarm can cause a message to be immediately relayed to a health care professional, emitting an auditory warning message via a speaker of the reusable pill dispenser 100, flashing illuminators of the reusable pill dispenser 100, vibrating the reusable pill dispenser 100, etc.

The CPU 106 can control the illuminating regions 103 of the reusable pill dispenser 100. In one or more examples, the CPU 106 can control the illuminating regions 103 based on information received from one or more of the sensor 108, the compliance mechanism 104, and the communication device 110. For example, the CPU 106 can control the illuminating regions 103 to illuminate when the patient is due to take a dose of the disposable blister package. In one or more examples, the CPU 106 can be configured to determine the patient's dosage regimen based on information received from one or more of the sensor 108 and the communication device 110. For instance, determining the dosage regimen can include determining which specific doses of a given disposable blister package should be taken at a time and determining the frequency of when those doses should be taken. The CPU 106 can be configured to control the illuminating regions 103 based on the determined dosage regimen, such as by illuminating the illuminating regions 103 when a dose is due and illuminating the apertures 102 which should be consumed at that time. The CPU 106 can include a memory to store instructions and/or information regarding dosage regimens, etc.

Optionally, the CPU 106 can determine the patient's dosage regimen based in part on a dosage database. For example, upon receiving information that the disposable blister package includes a particular medication, the CPU 106 can consult a dosage database for that particular medication that indicates the prescription for that particular medication. The CPU 106 can determine the patient's dosage regimen based on that prescription, and then control the illuminating regions 103 of the reusable pill dispenser 100 based on the dosage regimen. In one or more examples, the CPU 106 may communicate with an external device, such as the external device discussed above, via the communication device 110 to determine the patient's dosage regimen.

The sensor 108 of the reusable pill dispenser 100 can be configured to read an identifier of a disposable blister package. For instance, when the patient inserts a disposable blister package into the reusable pill dispenser 100, the sensor can read an identifier of the disposable blister package and convey the identifier to the CPU 106 of the reusable pill dispenser 100. The sensor 108 can be a reader, such as a radio frequency identification (RFID) reader, a near-field communication (NFC) reader, a barcode scanner, a camera, etc. In one or more examples, the CPU 106 can control the sensor 108 to read an identifier of a disposable blister package.

The communication device 110 can be a communication chip for communicatively connecting the reusable pill dispenser 100 to an external device, such as the external device of FIG. 1. The communication device 110 can be a wireless communication chip such as a near-field communication (NFC) chip or a Bluetooth chip. The communication device 110 can be configured for transmitting and receiving messages over one or more cellular networks, using short message service (SMS), over a Wi-Fi network, etc. The communication device 110 can be communicatively connected to the CPU 106 of the reusable pill dispenser 100 such that information received by the communication device 110 is conveyed to the CPU 106. In one or more examples, the communication device 110 can be included within the CPU 106.

In one or more examples, the communication device 110 can receive update information from an external device. For instance, the update information can be one or more of information relating to a device update, patient information update, and a dosage regimen update. The device update can include information to update software of the CPU 106 of the reusable pill dispenser 100. The patient information update can include, for example, information regarding the patient's prescribing physician, or information related to the patient's dosage regimen such as the patient's weight and age. The dosage regimen update can include information indicating that the patient's prescription has changed, for example indicating that the patient should take more doses or fewer doses of the medication or altering the timing or frequency of the doses. In one or more examples, the CPU 106 can control the illuminating regions 103 of the reusable pill dispenser 100 based on the update information received via the communication device 110. For instance, the CPU 106 may determine a revised dosage regimen for the patient based on the update information and then control the illuminating regions 103 of the reusable pill dispenser 100 based on the revised dosage regimen.

In one or more examples, the communication device 110 can convey adherence information to an external device. The adherence information can include information regarding whether the patient ingested the dose(s) of a disposable blister package within a predetermined period after the reusable pill dispenser 100 provided an alert to the patient that the dose(s) were due to be taken. The communication device 110 may convey adherence information based on the dosage regimen for a specific patient, such as by conveying adherence information after every dose is due to be taken. Alternatively, the communication device 110 may convey adherence information at regular intervals, such as once per day, once per week, etc.

In one or more examples, the reusable pill dispenser 100 also includes a display 112. The display 112 can present information such as the instructions for use of the medication in a blister package received by the reusable pill dispenser 100. Such information can include, for example, instructions to ingest the medication with food, to take the medication every 6 hours, to refrain from consuming alcohol while taking the medication, common side effects, etc. In one or more examples, the CPU 106 can control the information displayed on the display 112. For instance, the CPU 106 can continuously update a countdown timer shown on the display 112 that indicates the time remaining until the patient's next dose is due. The display 112 can be an E-ink display, a liquid crystal display (LCD), an LED display, etc.

The reusable pill dispenser 100 can include other components not shown in FIG. 2. For instance, the reusable pill dispenser 100 can include a battery. The battery may be a permanent battery or a replaceable battery. The reusable pill dispenser 100 can also include a mechanism to secure a disposable blister package within the reusable pill dispenser 100. Such mechanism can include, for example, clips, springs, etc. The reusable pill dispenser 100 can include a timer mechanism, (e.g., a mechanism that is part of the CPU 106 or separate from the CPU 106) for measuring the period between doses of a disposable blister package. The reusable pill dispenser 100 can be fabricated via a flexible material that flexes when pressed by a user. Optionally, the reusable pill dispenser can be fabricated via a mixture of rigid, semi-rigid, and flexible materials.

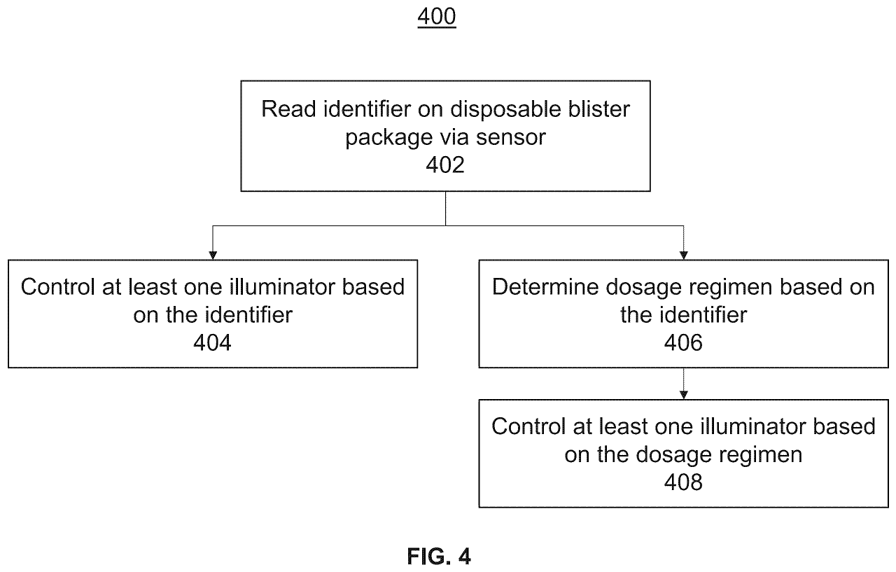

FIG. 3 shows an exemplary disposable blister package 200, according to one or more examples of the disclosure. The disposable blister package 200 can be received by a reusable pill dispenser such as the reusable pill dispenser discussed above and used in the smart package system described above. The disposable blister package 200 can include a number of domes 202 and an identifier 210.

The domes 202 (also called “blisters”) of the disposable blister package 200 can accommodate doses of medication in the form of capsules, tablets, or other solid dose drug product forms. The domes 202 of the disposable blister package may be formed from a transparent or opaque material, and may be formed of a frangible material that permits a dose of medication to be removed through the dome 722. Optionally, the domes 722 may be formed of a conductive material that can be broken by a user pressing a dose free, with the breaking of the dome 722 triggering a signal that a dose has been removed. Additionally or alternatively, the bottom surface of the disposable blister package 200 (e.g., opposing the respective domes 722) can include a frangible material, such as a foil, that a user can pierce by pressing downward on the dome 722 to force the dose to break the frangible material to remove the stored dose of medication. According to such embodiments, the entirety of the bottom surface can be formed of a frangible material, or else areas of frangible material can be provided in registration with the domes 722.

The doses within each dome 202 of the disposable blister package 200 can be accessed by a user pressing on a side of the disposable blister package 200 to encourage the dose to break free of the disposable blister package 200. For instance, a user may press directly on a dome 202 to press the dose free through an aperture or frangible seal (e.g., a thin layer of foil) on the underside of the disposable blister package 200. Alternatively, a user may press on the underside of the disposable blister package 200 to press the dose free through the dome 202 (i.e., through the blister). Accordingly, dome 202 and/or the portion of the disposable blister package 200 beneath each dome 202 may be formed of a frangible material (such as foil).

The identifier 210 of the disposable blister package 200 can be a label for the disposable blister package that can be read by a corresponding sensor, such as the sensor 108 of the reusable pill dispenser 100. The identifier 210 can contain information related to the disposable blister package 200. For example, the identifier 210 can include information such as one or more of a serial number for the disposable blister package 200, the name of the medication stored in the domes 202 of the disposable blister package 200, and an expiration date for the medication stored in the disposable blister package 200, patient information such as the patient's name and prescribing physician, and a pre-set dosage regimen or other prescription information. In one or more examples, the identifier 210 can be an RFID tag, an NFC tag or card, a barcode such as a quick response (QR) code, etc.

Referring now to both FIGS. 2 and 3, when a patient uses the reusable pill dispenser 100 with the disposable blister package 200, the user can insert the disposable blister package 200 into the reusable pill dispenser 100 such that the domes 202 align with the apertures 102 of the reusable pill dispenser 100. The apertures 102 of the reusable pill dispenser 100 can be sized such that they can receive the domes 202 of the disposable blister package 200 such that the domes 202 extend through the apertures 102 when the disposable blister package 200 is inserted into the reusable pill dispenser 100.

The reusable pill dispenser 100 is configured such that while the disposable blister package 200 is inserted into the reusable pill dispenser 100, a user may remove medication stored in the domes 202 of the disposable blister package 200. Where the apertures 102 extend through the reusable pill dispenser 100, the medication can be removed by a user pressing on a dome 202 to force the medication through the underside of the disposable blister package 200 and out the bottom of an aperture 102. Where the reusable pill dispenser 100 includes a top layer and a bottom layer with the apertures extending only through the top layer, the user can press on an underside of the bottom layer of the reusable pill dispenser 100 to encourage the dose stored within a dome 202 to break free of the material of the dome 202 and press the dose free of both the disposable blister package and the reusable pill dispenser 100. The underside of the reusable pill dispenser 100 may include a structure proximate to each aperture 102, such as a region formed of an inflexible material or containing a protrusion, that pushes the medication through the dome 202 when pressed by a user. Accordingly, a user can remove medication from the domes 202 of the disposable blister package 200 without removing the disposable blister package 200 from the reusable pill dispenser 100. As shown, the reusable pill dispenser 100 includes the same number of apertures 102 as the number of domes 202 of the disposable blister package 200. Optionally, the reusable pill dispenser 100 may include more apertures than the number of domes 202 of the disposable blister package 200.

After the patient inserts the disposable blister package 200 in the reusable pill dispenser 100, the reusable pill dispenser 100 can be configured to provide indications that assist the patient or HCP when taking medication stored in the disposable blister package 200. In one or more examples, the indications provided to the patient (or HCP) can be based on the identifier 210 of the disposable blister package 200, update information received by the communication device 110 from an external device, and/or the medication adherence information based on patient interaction with the compliance mechanism 104 of the reusable pill dispenser 100.

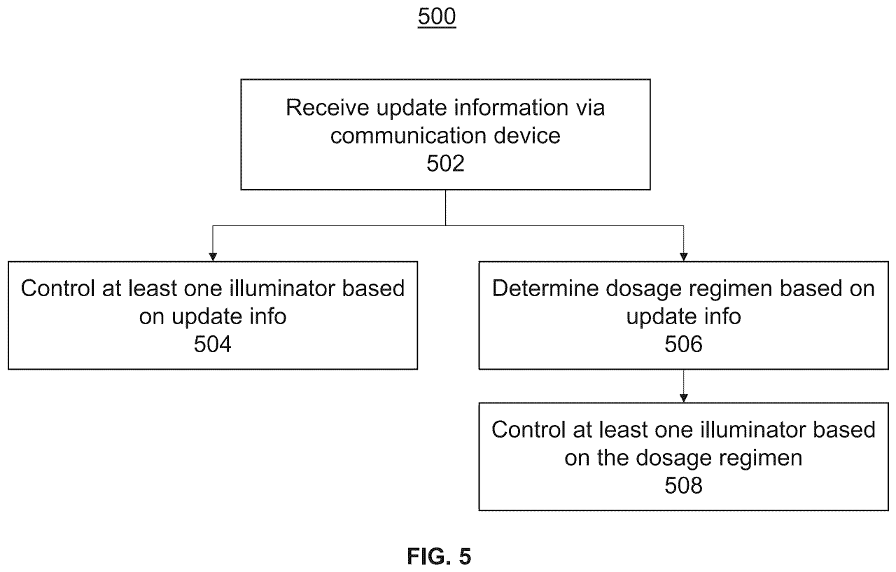

FIG. 4 shows an exemplary method 400 for administering medication via a reusable pill dispenser based on an identifier of a disposable blister package, according to one or more examples of the disclosure. The method 400 can be executed by a CPU of a reusable pill dispenser that holds a disposable blister package, such as the reusable pill dispenser 100 and disposable blister package 200 described above.

In one or more examples, the method 400 begins at step 402 wherein an identifier on a disposable blister package is read via a sensor. Reading an identifier at step 402 can involve a CPU of a reusable pill dispenser controlling a sensor, such as a radio frequency identification (RFID) reader, a near-field communication (NFC) reader, a barcode scanner, etc., to read a corresponding identifier, such as an RFID tag, an NFC tag or card, a barcode, etc. As discussed above, the identifier read at step 402 can contain information related to the disposable blister package, such as a serial number, medication name, expiration date, patient information, prescription information, a pre-set dosage regimen, etc.

In one or more examples, reading an identifier at step 402 can involve a CPU of a reusable pill dispenser controlling a camera to take one or more photos of an identifier on the disposable blister package. The photos of the identifier can be processed via an image recognition program of a computing device (such as the CPU 106 of FIG. 2 or an external device like the external device 30 of FIG. 1). For example, the CPU can capture a photo of the identifier and then process that using an image recognition program to access information associated with that specific identifier. When reading an identifier at step 402 via a camera, the CPU of the reusable pill dispenser may control the camera to read the identifier without taking one or more photos of the identifier.

After the identifier is read at step 402, the method 400 can move to step 404, wherein at least one illuminating region of a reusable pill dispenser is controlled based on the identifier. Controlling the illuminating region(s) of a reusable pill dispenser at step 404 can involve illuminating the illuminating region(s) associated with a given aperture of a reusable pill dispenser to provide a visual cue that the dose of the disposable blister package corresponding to that aperture/illuminating region is due to be taken. Accordingly, controlling the illuminating region(s) at step 404 can include providing a visual cue to the user (e.g., the patient) regarding which dose(s) of the disposable blister package should be consumed at that time, thereby assisting the user to take medication stored in the disposable blister package. In one or more examples, step 404 is performed closely after reading the identifier at step 402. For instance, step 404 may be performed within 5 seconds, 2 seconds, 0.5 seconds, etc., after reading the identifier. The information captured when reading the identifier may be saved in a memory (such as a memory of a CPU) and used to control the illuminating region(s) of the reusable pill dispenser at a later time. The information captured when reading the identifier may be saved in a memory and used to control the illuminating regions(s) closely after capturing the information and at a later time, as will be discussed below.

In one or more examples, when multiple doses of a disposable blister package are due to be taken at one time, controlling the illuminating region(s) at step 404 can involve illuminating each of the illuminating regions for which the dose in the aperture associated with those illuminating regions is due to be taken. For example, if a reusable pill dispenser has three apertures each with an associated illuminating region and each containing a dose of a disposable blister package and two of those doses are due at a certain time, controlling the illuminating region(s) at step 404 can involve illuminating the illuminating regions associated with the two apertures that contain the doses due at that time.

Controlling the illuminating region(s) based on the identifier read at step 402 can involve periodically controlling certain illuminating region(s) over an extended period. For instance, if the identifier read at step 402 is a pre-set dosage regimen indicating the patient should ingest one dose of the disposable blister package every 12 hours, controlling the illuminating region(s) based on that dosage regimen can involve illuminating one illuminating region of the reusable pill dispenser (e.g., the illuminating region associated with the aperture containing the dose of the disposable blister package that is due to be taken) every 12 hours, relying on a timer mechanism of the reusable pill dispenser to track the period between doses.

With respect to uptitration (gradual increase in a dose over time) and/or downtitration (gradual decrease in a dose over time) dosing regimens, controlling the illuminating region(s) can involve illuminating region increasing or decreasing numbers of illuminating regions each time the patient is due to consume a dose, according to the dosage regimen. Rather than a regimen where the patient increases/decreases the number of doses over time, an uptitration or downtitration regimen may involve the patient taking doses that contain different amounts of medication over time (e.g., dose strength). For instance, a patient may initially take one or more 10 mg doses, followed by 15 mg doses and then 20 mg doses, etc. Accordingly, controlling the illuminating region(s) can involve illuminating specific illuminating regions that are associated with the next dose according to the uptitration or downtitration dosage regimen.

Controlling the illuminating region(s) based on the identifier read at step 402 can involve controlling multiple illuminating regions at a given time. For instance, a disposable blister package may contain medication with two or more different active ingredients that should be taken together. Controlling the illuminating regions based on the identifier can thus involve simultaneously controlling the illuminating regions associated with the apertures containing such medication doses. Alternatively, the disposable blister package may contain medication with two or more active ingredients which should be consumed in a specific sequence according to a dosage regimen. Controlling the illuminating regions can thus involve controlling the illuminating regions associated with the doses according to that sequential dosage regimen.

Optionally, after the identifier is read at step 402, the method 400 can move to step 406, wherein a dosage regimen is determined based on the identifier. The reusable pill dispenser can include a program (stored locally or externally from a CPU of the reusable pill dispenser) configured to determine the dosage regimen for a specific drug based on information such as the serial number of the disposable blister package, a unique identifier corresponding to the patient, etc. For example, upon reading an identifier that indicates a particular medication is contained in the disposable blister package, the reusable pill dispenser can execute a program that determines the appropriate dosage regimen based on that particular medication.

Optionally, determining the dosage regimen at step 406 can involve consulting a dosage database. For instance, upon reading an identifier such as a serial number, the reusable pill dispenser can consult a dosage database associated with that serial number to determine the patient's dosage regimen (e.g., how many doses due at what frequency). In one or more examples, the reusable pill dispenser can communicate with an external device via a communication chip to determine the dosage regimen.

After the dosage regimen is determined at step 406, the method 400 can move to step 408 wherein at least one illuminating region of a reusable pill dispenser is controlled based on the dosage regimen. Controlling the illuminating region(s) of the reusable pill dispenser at step 408 can be performed as described above with respect to step 404. For example, controlling the illuminating region(s) of a reusable pill dispenser at step 408 can involve illuminating the illuminating region(s) associated with a given aperture of a reusable pill dispenser to provide a visual cue that the dose of the disposable blister package corresponding to that aperture/illuminating region is due to be taken, periodically controlling certain illuminating region(s) over an extended period, and/or illuminating each of the illuminating regions for which the dose in the aperture associated with those illuminating regions is due to be taken when multiple doses are due to be taken at one time.

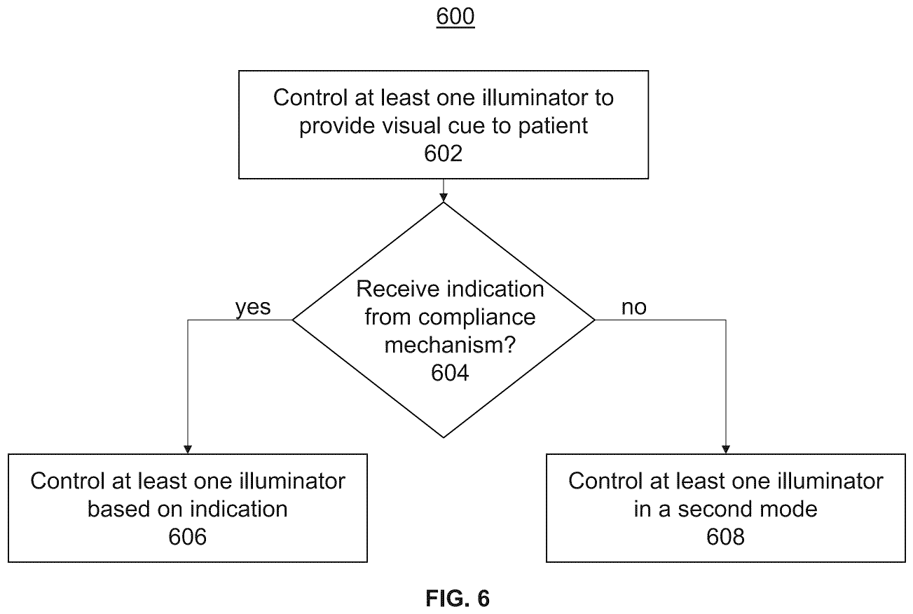

FIG. 5 shows an exemplary method 500 for administering medication via a reusable pill dispenser based on received update information, according to one or more examples of the disclosure. The method 500 can be executed by a CPU of a reusable pill dispenser that holds a disposable blister package, such as the reusable pill dispenser 100 and disposable blister package 200 described above.

In one or more examples, the method 500 begins at step 502 wherein update information is received. The update information can be received via a communication chip, such as the communication device 110 of the reusable pill dispenser 100 of FIG. 2, from an external device, such as the external device 30 of FIG. 1. As discussed above, exemplary update information includes a patient information update, which may include information relevant to the patient's dosage regimen such as the patient's weight and age, and/or a dosage regimen update, which can include information that indicates the patient's prescription has been updated.

After the update information is received at step 502, the method 500 can move to step 504, wherein at least one illuminating region of a reusable pill dispenser is controlled based on the update information. Controlling the illuminating region(s) of the reusable pill dispenser at step 504 can be performed as described above. For example, controlling the illuminating region(s) of a reusable pill dispenser can involve illuminating the illuminating region(s) associated with a given aperture of a reusable pill dispenser to provide a visual cue that the dose of the disposable blister package corresponding to that aperture/illuminating region is due to be taken, periodically controlling certain illuminating region(s) over an extended period, and/or illuminating each of the illuminating regions for which the dose in the aperture associated with those illuminating regions is due to be taken when multiple doses are due to be taken at one time. When the update information received at step 502 indicated the patient's prescription was updated, controlling the illuminating region(s) at step 504 can involve controlling the illuminating region(s) to provide visual cue(s) to the patient corresponding to the updated prescription.

Optionally, after the update information is received at step 502, the method 500 can move to step 506, wherein a dosage regimen is determined based on the update information. For example, if the update information received at step 502 indicates the patient's weight has changed, determining a dosage regimen at step 506 can include determining the appropriate number of doses the patient should take at a given dosing period based on the patient's new weight to generate a revised dosage regimen.

After the dosage regimen is determined at step 506, the method 500 can move to step 508 wherein at least one illuminating region of a reusable pill dispenser is controlled based on the dosage regimen. Controlling the illuminating region(s) of the reusable pill dispenser at step 508 can be performed as described above. For example, controlling the illuminating region(s) of a reusable pill dispenser can involve illuminating the illuminating region(s) associated with a given aperture of a reusable pill dispenser to provide a visual cue that the dose of the disposable blister package corresponding to that aperture/illuminating region is due to be taken, periodically controlling certain illuminating region(s) over an extended period, and/or illuminating each of the illuminating regions for which the dose in the aperture associated with those illuminating regions is due to be taken when multiple doses are due to be taken at one time.

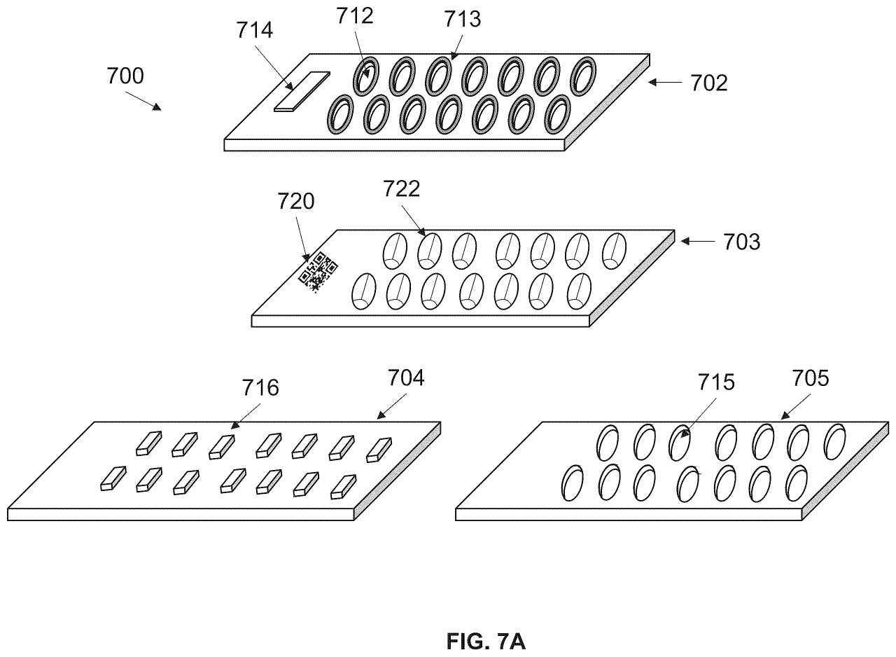

FIG. 6 shows an exemplary method 600 for administering medication via a reusable pill dispenser based on a received indication, according to one or more examples of the disclosure. The method 600 can be executed by a CPU of a reusable pill dispenser that holds a disposable blister package, such as the reusable pill dispenser 100 and disposable blister package 200 described above.

In one or more examples, the method 600 begins at step 602, wherein at least one illuminating region of the reusable pill dispenser is controlled to provide a visual cue to the patient that one or more doses are due to be taken. For instance, the reusable pill dispenser may illuminate the illuminating regions associated with the apertures containing the doses of the disposable blister package that are due, to indicate to the patient that they should take those doses. Controlling the illuminating region(s) of the reusable pill dispenser at step 602 can be performed as described above. For example, controlling the illuminating region(s) of a reusable pill dispenser can involve illuminating the illuminating region(s) associated with a given aperture of a reusable pill dispenser to provide an indication that the dose of the disposable blister package corresponding to that aperture/illuminating region is due to be taken, periodically controlling certain illuminating region(s) over an extended period, and/or illuminating each of the illuminating regions for which the dose in the aperture associated with those illuminating regions is due to be taken when multiple doses are due to be taken at one time. Optionally, controlling the illuminating regions at step 602 of the method 600 can be based on an identifier read from a disposable blister package as discussed in method 400, or based on update information received via a chip as discussed in method 500.

After providing the visual cue to the patient by controlling the illuminating regions at step 602, the method can move to step 604 wherein the reusable pill dispenser determines whether an indication from a compliance mechanism is received. Receiving an indication at step 604 can indicate that the reusable pill dispenser received confirmation from a user that the user took the dose(s) of the disposable blister pack that the reusable pill dispenser indicated the user should take by providing visual cues at step 602. Receiving an indication at step 604 can involve receiving a user input, such as a user activation of a compliance mechanism. For instance, if the compliance mechanism is a push button or switch, receiving the indication at step 604 can involve the user pressing the button or activating the switch. If the compliance mechanism is an electrical trigger (such as a breakable wire, electrical trace, or thin film circuit), the indication may be received automatically when the user removes a dose from an aperture of the reusable pill dispenser.

If at step 604 the reusable pill dispenser determines an indication was received, the method 600 can move to step 606 wherein at least one illuminating region is controlled based on the indication. For example, controlling the illuminating region(s) after receiving the indication can involve turning off the illuminating region(s) of the reusable pill dispenser, to provide a visual cue to the patient that they successfully took the dose of the disposable blister package as required.

If at step 604 the reusable pill dispenser determines an indication was not received, the method 600 can move to step 608 wherein at least one illuminating region is controlled in a second mode. Different modes of controlling the illuminating regions of the reusable pill dispenser can include illuminating the illuminating regions, flashing the illuminating regions, illuminating the illuminating regions in different colors, turning off the illuminating regions, etc. In one or more examples, the reusable pill dispenser controls the illuminating regions in a first mode (e.g., at step 602 to provide a visual cue to the patient), and if a predetermined period passes without receiving an indication, controls the illuminating regions in a second mode. For instance, to provide the visual cue at step 602, the reusable pill dispenser may control the illuminating regions in a first mode by illuminating certain illuminating regions, and then if a predetermined period passes without receiving an indication, the reusable pill dispenser may control the illuminating regions in a second mode by flashing those same illuminating regions. The predetermined period can be 5 seconds, 10 seconds, or more or less than 5 or 10 seconds. Upon failing to receive an indication from the user for a predetermined period, such as after 2 minutes, the reusable pill dispenser may turn off the illuminating regions.

Optionally, if no indication is received at step 604, the reusable pill dispenser may communicate this via a chip to an external device. Failure to receive an indication may indicate medication non-adherence (e.g., that the patient skipped a dose of the disposable blister package). For clinical trials or certain drugs where medication adherence is particularly sensitive, communicating medication non-adherence to an external device may include alerting the prescribing physician or trial administrator so that they can contact the patient.

Optionally, if no indication is received at step 604, the reusable pill dispenser may determine an updated dosage regimen. For instance, a particular drug may require strict adherence regarding when doses are ingested and may require updating the timing of subsequent doses if a particular dose is missed. Accordingly, the reusable pill dispenser may calculate this update upon failing to receive an indication at step 604.

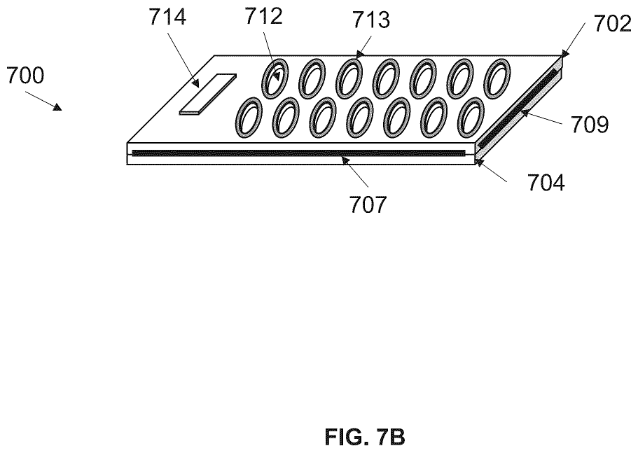

FIG. 7A shows an exploded view of an exemplary reusable pill dispenser 700, according to one or more examples of the disclosure. The reusable pill dispenser 700 stores a disposable blister package 703 between a top portion 702 and a bottom portion 704 or bottom portion 705. The reusable pill dispenser 700 may include multiple “top” layers and/or multiple “bottom layers. In one or more examples, the reusable pill dispenser 700 can implement one or more of method 400 of FIG. 4, method 500 of FIG. 5, and method 600 of FIG. 6.

The disposable blister package 703 includes a plurality of domes 722 containing medication and an identifier 720, which in this example includes a QR code. Like the domes 202 of the disposable blister package 200 of FIG. 3, the domes 722 of the disposable blister package 703 can contain medication that can be removed from the disposable blister package 703 by pressing the dome 722 to force medication through an underside of the disposable blister package 703 or by pressing the underside of the disposable blister package 703 to force medication out through the dome 722. Similarly, the identifier 720 can function like the identifier 210 of the disposable blister package 200 of FIG. 3, and can contain information related to the medication of the disposable blister package 703.

The top portion 702 includes a user compliance mechanism, which in this example is a button 714. The button 714 can accept an indication from a user that the user has taken a dose of the disposable blister package 703. The top portion 702 also includes a number of apertures 712 each surrounded by an illuminating region 713. Additionally, the top portion and/or bottom portion 704 of the reusable pill dispenser 700 can include a CPU (not shown) that controls the illuminating regions 713 and is communicatively connected to or can control a sensor (not shown) and a chip (not shown). The CPU, sensor, and chip can function like the CPU 106, the sensor 108 and the communication device 110 of the reusable pill dispenser 100 of FIG. 2. For instance, the sensor of the reusable pill dispenser 700 can read the identifier 720 on the disposable blister package 703 and convey the identifier to the CPU of the reusable pill dispenser 700, and the CPU can control the illuminating regions 713 based on the identifier 720.

In one or more examples, the entirety of the top portion 702, or a portion of the top portion 702, may be a screen that includes distinct regions that can be illuminated. For instance, the top portion 702 can include a screen with apertures 712 with integrated illuminators (such as LEDs or OLEDs) located in regions that are adjacent to the apertures 712. These integrated illuminators can form the illuminating regions 713 surrounding the apertures 712.

The reusable pill dispenser 700 may incorporate the bottom portion 704 or the bottom portion 705. The bottom portion 704 comprises a plurality of structures 716 that are configured to align with each dome 722 of the disposable blister package 703 when the reusable pill dispenser 700 is assembled. The structures 716 may be formed from a region of inflexible material, or as shown here a protrusion that extends from the bottom portion 704. The structures 716 can be configured such that they can help to push a dose of medication contained within a dome 722 free from the reusable pill dispenser 700.

The structures 716 may be configured to help push a dose of medication free through the dome 722 when a user presses against the underside of the bottom portion 704 in the region corresponding to the structure 716 and the dome 722 containing the dose. In such scenario, the disposable blister package 703 can be inserted into the reusable pill dispenser 700 such that the domes 722 of the blister package protrude from the disposable blister package 703 upwardly as shown in FIG. 7A and extend through the apertures 712 of the top portion 702. Alternatively, the structures 716 may be configured to help push a dose of medication free through the underside of the disposable blister package 703 (such as through a perforated seal or thin region of foil) when a user presses against the underside of the bottom portion 704 in the region corresponding to the structure 716 and the dome 722 containing the dose. In this scenario, the disposable blister package 703 may be inserted into the reusable pill dispenser 700 such that the domes 722 of the blister package protrude from the disposable blister package 703 downwardly (i.e., the disposable blister package 703 is flipped over), with the regions on the underside of the disposable blister package 703 adjacent to each dome 722 aligned with the apertures 712 of the top portion such that a given structure 716 contacts a dome 722 of the disposable blister package 703. When a user presses against the underside of the bottom portion 704 in the region corresponding to the structure 716 and the dome 722 containing the dose, the medication within the dome 722 is forced out of the underside of the disposable blister package and through the aperture 712 adjacent to the dome 722.

In contrast to the bottom portion 704, the bottom portion 705 includes a plurality of apertures 715 that are configured to align with each dome 722 of the disposable blister package 703 when the reusable pill dispenser 700 is assembled. In this configuration, a user can push a dose of medication contained within a dome 722 free through the bottom of the disposable blister package 703 and through the corresponding aperture 715 of the bottom portion 705 when a user presses against the dome 722 containing the dose.

The top portion 702 of the reusable pill dispenser 700 connects to the bottom portion 704. In one or more examples, the top portion 702 connects to the bottom portion 704 via one or more of press fitting, snap fitting, screws, adhesive, Velcro, welding, riveting, adhesion bonding, etc. In one or more examples, the top portion 702 and bottom portion 704 are fixedly connected to one another, such as via welding, riveting, adhesion bonding, etc. In one or more examples, the top portion 702 and bottom portion 704 are releasably connected to one another. For instance, the top portion 702 can include studs that mate with hubs of the bottom portion 704 (or vice versa) via an interference fit. In one or more examples, the top portion 702 may connect to the bottom portion 704 via a living hinge on one side with one or more latches on the opposing side (e.g., similar to a wallet).

When the top portion 702 and the bottom portion 704 are releasably connected to one another, a user may insert the disposable blister package 703 into the reusable pill dispenser 700 by separating the top portion 702 and the bottom portion 704 and placing the disposable blister package 703 in the appropriate location (e.g., with the domes 722 aligned with the apertures 712 of the top portion 702), and then reconnecting the top portion 702 and the bottom portion 704.

When the top portion 702 and the bottom portion 704 are fixedly connected to one another, the reusable pill dispenser 700 can include a slot for a user to insert the disposable blister package 703 into the reusable pill dispenser 700. This is shown in FIG. 7B, which depicts a perspective view of an assembled reusable pill dispenser 700, according to one or more examples of the disclosure. As shown, the reusable pill dispenser 700 includes a side slot 707 along the side of the top portion 702 and the bottom portion 704, and an end slot 709 along another side of the top portion 702 and the bottom portion 704. The reusable pill dispenser 700 may include one or both of the side slot 707 and the end slot 709, which can receive the disposable blister package 703 when inserted by a user. Accordingly, the height and length of the side slot 707 and/or the end slot 709 can be sized to receive the disposable blister package 703 (e.g., height sized to accommodate the height of the disposable blister package 703 including the domes 722 and length sized to accommodate the length of the disposable blister package 703).

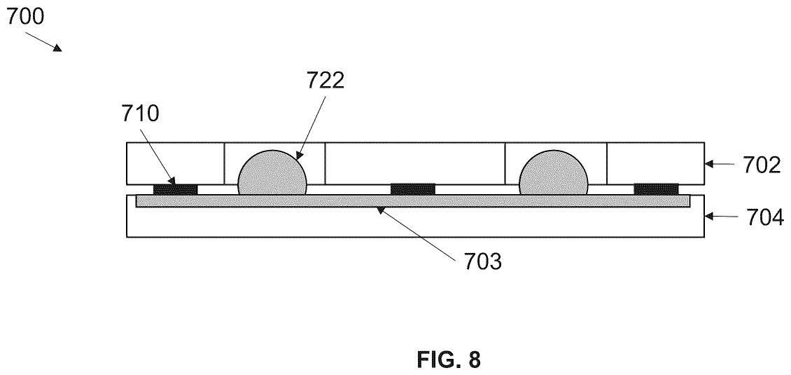

Once the disposable blister package 703 is inserted into the reusable pill dispenser 700, the reusable pill dispenser 700 can include one or more elements to ensure the domes 722 of the disposable blister package 703 remain aligned with the apertures 712 of the reusable pill dispenser 700. This is shown more clearly in FIG. 8, which shows a cutaway side view of the exemplary reusable pill dispenser 700 of FIG. 7A when assembled. As shown in FIG. 8, the disposable blister package 703 is secured between the top portion 702 and the bottom portion 704 of the reusable pill dispenser 700 via a number of securing portions 710. In one or more examples, the securing portions 710 can be attached to the underside of the top portion 702 such that when the top portion 702 connects to the bottom portion 704, the securing portions 710 press the disposable blister package 703 between the top portion 702 and the bottom portion 704 to fix the disposable blister package 703 in place and ensure the domes 722 remain aligned with the apertures of the top portion 702. The securing portions 710 can be one or more of protrusions, springs, clips, tension springs, etc. The securing portions 710 may be located as shown in FIG. 8, with three separate securing portions 710, or may be dispersed around the perimeter of the reusable pill dispenser 700, such as around the edges of the disposable blister package 703 along the top portion 702 or the bottom portion 704. The reusable pill dispenser 700 may include only one securing portion 710, or more than the three securing portions 710 shown in FIG. 8.

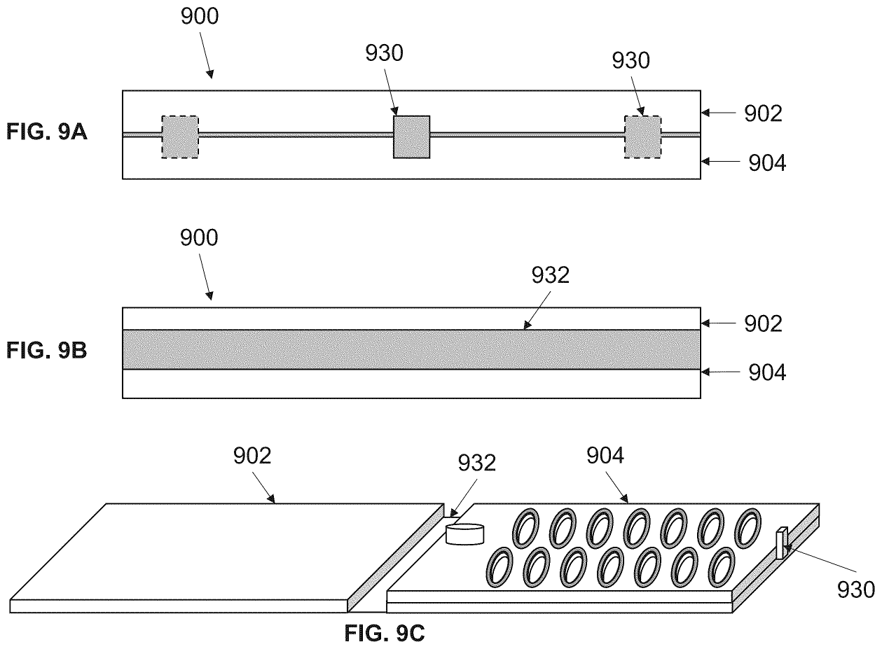

In one or more examples, the reusable pill dispenser 700 can be configured to open and close like a wallet, with the medication located in the domes 722 of the disposable blister package 703 only accessible when the wallet is open. An exemplary wallet-shaped reusable pill dispenser is shown in FIGS. 9A and 9B which show a first side view of an exemplary reusable pill dispenser 900 in a closed configuration and an opposing side view of the reusable pill dispenser 900, respectively. As shown in FIG. 9A, the reusable pill dispenser 900 includes one or more latches 930 between a top portion 902 and a bottom portion of 904. The latches 930 can latch the top portion 902 and the bottom portion 904 of the reusable pill dispenser 900 together when the reusable pill dispenser is in the closed configuration. On the opposite side, as shown in FIG. 9B, the reusable pill dispenser 900 includes a hinge 932, such as a living hinge, that connects the top portion 902 to the bottom portion 904.

FIG. 9C illustrates a perspective view of the reusable pill dispenser 900 when opened. When opening the reusable pill dispenser 900, the top portion 902 rotates about the hinge 932 like a wallet, providing access to the doses of a disposable blister package within the reusable pill dispenser 900. When opened, the reusable pill dispenser 900 can be configured as shown in FIG. 7B with illuminating regions located proximate to apertures that align with the domes of a disposable blister package.

In one or more examples, the reusable pill dispenser 900 can be sized to fit within a pocket when in the closed configuration. For example, when closed, the reusable pill dispenser 900 can be around 4 inches long and 4 inches wide. Optionally, the closed shape of the reusable pill dispenser 900 is square, such as 4 inches by 4 inches, 5 inches by 5 inches, 6 inches by 6 inches, etc. In one or more examples, the closed shape of the reusable pill dispenser 900 is rectangular, such as 4 inches by less than or more than 4 inches. In one or more examples, the reusable pill dispenser 900 is larger than around 4 inches by 4 inches. The reusable pill dispenser 900 may be, for example, 3×6 inches, 4×6 inches, 4×7 inches, etc. The size of the reusable pill dispenser 900 may be based on the number of pills for a particular dosing regimen. For instance, reusable pill dispenser 900 for a dosing regimen with 8 pills may be smaller than a reusable pill dispenser 900 for a dosing regimen with 20 pills. The overall thickness of the reusable pill dispenser may be, for example, 0.4 inches, 0.5 inches, 0.6 inches, 1 inch, 2 inches, 3 inches, etc.

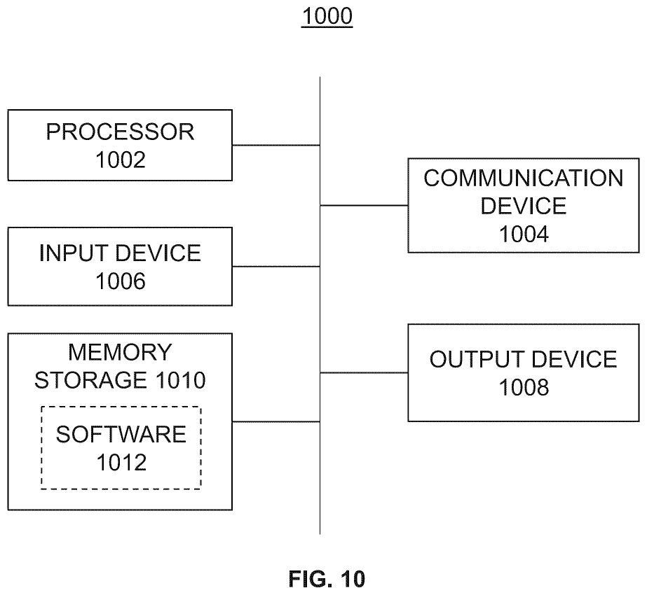

FIG. 10 illustrates an example of a computing device 1000, according to one or more examples of the disclosure. In one or more examples, the device 1000 can be implemented as the CPU 106 of the reusable pill dispenser 100 of FIG. 2 and/or the external device 30 of FIG. 1. Device 1000 can be a host computer connected to a network. Device 1000 can be a client computer or a server. As shown in FIG. 10, device 1000 can be any suitable type of microprocessor-based device, such as a personal computer, workstation, server, or handheld computing device (portable electronic device) such as a phone or tablet. The device can include, for example, one or more of processors 1002, input device 1006, output device 1008, storage 1010, and communication device 1004. Input device 1006 and output device 1008 can generally correspond to those described above and can be either connectable or integrated with the computer.

Input device 1006 can be any suitable device that provides input, such as a touch screen, keyboard or keypad, mouse, user compliance mechanism, or voice-recognition device. Output device 1008 can be any suitable device that provides output, such as a touch screen, haptics device, illuminating regions, or speaker.

Storage 1010 can be any suitable device that provides storage, such as an electrical, magnetic, or optical memory, including a RAM, cache, hard drive, or removable storage disk. Communication device 1004 can include any suitable device capable of transmitting and receiving signals over a network, such as a network interface chip or device. The components of the computer can be connected in any suitable manner, such as via a physical bus or wirelessly.

Software 1012, which can be stored in storage 1010 and executed by processor 1002, can include, for example, the programming that embodies the functionality of the present disclosure (e.g., as embodied in the devices as described above).

Software 1012 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1010, that can contain or store programming for use by or in connection with an instruction execution system, apparatus, or device.

Software 1012 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 1000 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1000 can implement any operating system suitable for operating on the network. Software 1012 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A reusable pill dispenser for assisting a user to take medication stored in a disposable blister package, the reusable pill dispenser comprising:
a plurality of apertures, wherein each of the plurality of apertures is arranged to align with a dose of the disposable blister package;
a plurality of illuminating regions, wherein at least one of the plurality of illuminating regions is associated with at least one of the plurality of apertures;
a sensor to read an identifier on the disposable blister package; and
a controller configured to control the at least one of the plurality of illuminating regions that is associated with the at least one of the plurality of apertures based on the identifier.

2. The reusable pill dispenser of claim 1, wherein the controller is configured to determine a dosage regimen based on the identifier.

3. The reusable pill dispenser of claim 2, wherein the controller is configured to control the at least one of the plurality of illuminating regions that is associated with the at least one of the plurality of apertures based on the determined dosage regimen.

4. The reusable pill dispenser of claim 2, wherein the dosage regimen is based in part on a dosage database corresponding to the identifier.

5. The reusable pill dispenser of claim 1, comprising a user compliance mechanism for accepting an indication that the user has taken at least one dose of the disposable blister package.

6. The reusable pill dispenser of claim 5, wherein the controller is configured to control the at least one of the plurality of illuminating regions that is associated with the at least one of the plurality of apertures based on the indication.

7. The reusable pill dispenser of claim 6, wherein controlling the at least one of the plurality of illuminating regions that is associated with the at least one of the plurality of apertures comprises:
controlling the at least one of the illuminating regions that is associated with the at least one of the plurality of apertures in a first mode; and
if a predetermined period passes without receiving the indication, controlling the at least one of the illuminating regions that is associated with the at least one of the plurality of apertures in a second mode.

8. The reusable pill dispenser of claim 5, wherein the indication specifies which doses of the disposable blister package the user has taken and the controller is configured to trigger an alarm if the at least one dose of the disposable blister package the user has taken is contrary to a dosage regimen for the user.

9. (canceled)

10. The reusable pill dispenser of claim 1, comprising a communication device to communicate with an external device.

11. The reusable pill dispenser of claim 10, wherein the communication device is operative to receive update information from the external device related to one or more of a device update, patient information update, and a dosage regimen update.

12. The reusable pill dispenser of claim 11, wherein the controller is configured to determine an updated dosage regimen based at least in part on the update information.

13. The reusable pill dispenser of any claim 10, wherein the communication device is configured for transmitting and receiving messages using i) one or more cellular networks, ii) short message service (SMS), and/or iii) Wi-Fi.

14. (canceled)

15. (canceled)

16. The reusable pill dispenser of claim 1, wherein the identifier comprises one or more of a serial number for the disposable blister package, a name of the medication stored in the disposable blister package, patient information corresponding to the user, an expiration of the medication stored in the disposable blister package, and a prescription for the medication stored in the disposable blister package.

17. The reusable pill dispenser of claim 1, wherein controlling the at least one of the plurality of illuminating regions that is associated with the at least one of the plurality of apertures comprises illuminating the at least one illuminating region when a dose of the disposable blister package is due to be taken.

18. (canceled)

19. (canceled)

20. (canceled)

21. The reusable pill dispenser of claim 1, comprising a top layer that comprises the plurality of apertures and the plurality of illuminating regions, wherein each of the plurality of illuminating regions are positioned on the top layer adjacent to a respective one of the plurality of apertures.

22. The reusable pill dispenser of claim 1, comprising a display for communicating information to the user.

23. (canceled)

24. (canceled)

25. The reusable pill dispenser of claim 1, comprising a first portion and a second portion connected by a hinge, wherein the first portion and the second portion contact one another in a closed position.

26. The reusable pill dispenser of claim 24, comprising a latch to secure the reusable pill dispenser in the closed position.

27. The reusable pill dispenser of claim 1, comprising a plurality of spring clips to retain the disposable blister package inside the reusable pill dispenser.

28. A method for assisting a user to take medication stored in a disposable blister package using a reusable pill dispenser, the method comprising:
reading an identifier on the disposable blister package via a sensor of the reusable pill dispenser, the reusable pill dispenser comprising a plurality of apertures, wherein each of the plurality of apertures is arranged to align with a dose of the disposable blister package; and
controlling at least one illuminating region of a plurality of illuminating regions of the reusable pill dispenser based on the identifier, wherein at least one illuminating region of the plurality of illuminating regions is associated with at least one of a plurality of apertures of the reusable pill dispenser.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)
